(19) United States
(12) Patent Application Publication
(10) Pub. No.: US 2024/0245307 A1
(43) Pub. Date: Jul. 25, 2024

(54) MULTI-SENSOR UPPER ARM BAND FOR PHYSIOLOGICAL MEASUREMENTS AND ALGORITHMS TO PREDICT GLYCEMIC EVENTS

(71) Applicant: THE TEXAS A&M UNIVERSITY SYSTEM, College Station, TX (US)

(72) Inventors: Kimberly BRANAN, College Station, TX (US); Cody LEWIS, College Station, TX (US); Richard HORNER, College Station, TX (US); Gerard L. COTE, College Station, TX (US); Darpit DAVE, College Station, TX (US); Kathan VYAS, College Station, TX (US); Madhav ERRAGUNTLA, College Station, TX (US); Ricardo GUTIERREZ-OSUNA, College Station, TX (US); Justin MCMURRAY, College Station, TX (US)

(21) Appl. No.: 18/418,861

(22) Filed: Jan. 22, 2024

Related U.S. Application Data
(60) Provisional application No. 63/440,221, filed on Jan. 20, 2023.

(51) Int. Cl. A61B 5/0205; A61B 5/00; A61B 5/01; A61B 5/0295; A61B 5/0533; A61B 5/08; A61B 5/11; A61B 5/282; A61B 5/346

(52) U.S. Cl. CPC A61B 5/02055; A61B 5/11; A61B 5/282; A61B 5/6824; A61B 5/7235; A61B 5/7275; A61B 5/7282; A61B 5/01; A61B 5/0295; A61B 5/0533; A61B 5/08; A61B 5/346; A61B 2562/0219; A61B 2562/0233; A61B 2562/0271

(57) ABSTRACT

A wearable multi-sensor device for measuring physiological properties includes a plurality of non-invasive sensors, such as a single-sided electrocardiography sensor, a bioimpedance and electrodermal activity sensor, a skin temperature sensor, and a photoplethysmography sensor. The device is configured to secure a skin-facing side of the sensors to exposed skin of a user and includes a communication module configured to receive signals from the plurality of non-invasive sensors and output data from the device, the being suitable for use in predicting glycemic events in the user.

### CROSS-REFERENCE TO RELATED APPLICATIONS

This application claims priority to and the benefit of U.S. Provisional Application Ser. No. 63/440,221, entitled “MULTI-SENSOR UPPER ARM BAND FOR PHYSIOLOGICAL MEASUREMENTS AND ALGORITHMS TO PREDICT GLYCEMIC EVENTS,” and filed Jan. 20, 2023, the contents of which is incorporated by reference herein in its entirety.

### GOVERNMENT RIGHTS

This invention was made with government support under Award Number 2037383 provided by The National Science Foundation. The government has certain rights in the invention.

### FIELD

The present disclosure relates to wearable multi-sensor devices for measuring biosignals related to physiological events and associated methods for predicting glycemic events.

### BACKGROUND

Diabetes and other diseases affecting glucose are important disease states, often requiring constant monitoring in patients for adequate treatment. In this regard, current practices for accurate monitoring of glucose require invasive techniques that are often undesirable in patients. As such, new mechanisms for monitoring glucose in patients and reporting glycemic events associated with hyperglycemia or hypoglycemia are highly desirable.

Over 30 million people in the United States have diabetes and 1.5 million new diagnosis are made each year, with an estimated annual cost exceeding $300B. Managing diabetes requires balancing the long-term risks of diabetes complication related to hyperglycemia (e.g., blindness, kidney failure, amputation, stroke, and heart disease) and the acute risks of hypoglycemia (e.g., seizure, coma, or death). Because the short-term consequences can result in immediate disability and death, the fear of hypoglycemia often leads to less aggressive insulin therapy, increasing the patient's long-term exposure to hyperglycemia and risk of debilitating complications in later life. Thus, accurate detection and tracking of time in the hyperglycemia range is important for effective prevention of long-term complications associated with diabetes. At present, however, this can only be achieved with continuous glucose monitors (CGMs), which are expensive, invasive, and typically only prescribed to 5% of the patients with diabetes (type 1). Thus, low-cost noninvasive wearable sensors, and the associated machine-learning algorithms to predict hypoglycemic events and characterize hyperglycemic time in range, would be game changers to manage both short and long term complications associated with diabetes.

A number of physiological variables have been investigated as potential indicators of hypoglycemic and hyperglycemic events. Early work focused on skin temperature and skin conductivity, which were found to decrease at the onset of hypoglycemia. Several commercial instruments were developed in the early 1980s, but they suffered from a number of issues, such as false alarms due to perspiration that is unrelated to hypoglycemia and missed alarms in patients who do not experience these symptoms due to hypoglycemia unawareness. Electroencephalography (EEG) has also been used as a potential indicator of hypoglycemia, with early work from the 1950s showing that hypoglycemia is associated with increases in delta and gamma frequencies and decreases in alpha frequency, and more recent work indicating that hypoglycemia is associated with a decrease in signal complexity. EEG measurements are far more involved than skin temperature/conductivity, but improvements in sensing technology may make the approach more practical in the future. Changes in cardiac output have been associated with hypoglycemia, most notably a lengthened QT interval and a reduction in heart rate variability (HRV), and these effects are inverted in hyperglycemia, which suggests that electrocardiography (ECG) measurements could be used to detect both types of events. As a whole, these studies indicate that glucose excursions from their normal range induce a variety of changes on physiological signals that can be measured non-invasively.

Most prior work focused on using non-invasive physiological sensors to replace direct glucose measurements. However, some studies have examined whether these physiological signals could be used to complement glucose measurements from CGMs (e.g., using CGM and ECG measurements to predict spontaneous hypoglycemia in during normal daily activities) and their results support the position that, even when CGMs are used, measuring additional physiological signals can improve the prediction of hypoglycemic events.

### SUMMARY

Current methods to forecast hypoglycemia and hyperglycemia rely on direct measurement of glucose from blood samples, which requires invasive sensors such as continuous glucose monitors (CGMs). In contrast, examples of the present disclosure predict glycemic events indirectly, by analyzing changes in physiological signals that can be measured non-invasively. Prior efforts in this area have only focused on a single sensing modality, primarily electrocardiography. In contrast, examples of the present disclosure can information from multiple independent sensing modalities, conferring a detection system with a high degree of fault-tolerance and robustness to motion artifacts in an unobtrusive form factor. Further, examples include the use of multiple sensing modalities that enable extraction of other physiological variables, such as combined electrocardiography and photoplethysmography for estimating blood pressure, which is very significant for other conditions like hypertension and congestive heart failure. Examples also include machine-learning techniques for predicting not only impending hypoglycemic events at multiple time horizons, but also characterizing time-in-range for hyperglycemia. Further, examples include the development and training of deep-learning model extract glucose-related information automatically from raw sensor signals using convolutional neural networks, and techniques to learn temporal patterns using recurrent neural networks.

Certain examples of the present disclosure provides for armband device that can provide a noninvasive system for monitoring glucose and glycemic events. Furthermore, examples of the armband devices can be utilized to alert patients, caregivers, and healthcare providers with events of hyperglycemia or hypoglycemia.

Example armband devices can include various sensors and can be positioned on the upper arm of a wearer. For instance, example armband devices can include integrated sensors and accompanying circuitry to measure photoplethysmography (PPG), bioimpedance (BioZ), single-sided electrocardiogram (SS-ECG), electrodermal activity (EDA), and temperature. In addition, an accelerometer and gyroscope can also be included to provide contextual awareness to activity of the user, such as exercising. The accelerometer and gyroscope can also be included to help mitigate noise due to macro-motion artifacts from the collected biosignals.

As described herein, examples include the use of PPG to measure a change in the blood volume of the probed tissues. Examples include the use of BioZ can be used to measure a change in impedance due to changes in cellular shapes and orientations which occurs with a pulse propagating through the blood vessels. Furthermore, examples include the use of SS-ECG to measure an electrical biopotential from the heart, which occurs with every beat of the heart. Examples include the use of these three modalities for predicting glycemic events in additional to being used for various cardiovascular diseases like hypertension via calculation of heart rate, heart rate variability, respiration rate, and potentially cuffless blood pressure. In addition, examples include the use of EDA to provide a measure of skin conductance and temperature as a measure of skin temperature fluctuations, which can, in some examples, assist in the prediction of glycemic events as well as being used to determine emotional state or physiological stimulus (e.g., stress). Examples include the use of an accelerometer and/or a gyroscope to determine the position of the device relative to the user and provide contextual awareness to determine if the user is in a state of motion or is sedentary.

Finally, examples include armband devices that utilize algorithms to provide noninvasive means for prediction of hypoglycemic and hyperglycemic events. Moreover, examples include devices configured for monitoring cardiovascular diseases such as hypertension or for predicting emotional state or stress.

Examples of the present disclosure also include predictive models, including associated training and development techniques, of glycemic events. Examples include statistical and deep-learning models that are configured to identify hypoglycemic and hyperglycemic events by analyzing subtle changes in physiology using, for examples, any of the sensing techniques disclosed herein, including ECG and PPG.

Additional features of the present disclosure will become apparent to those skilled in the art upon consideration of illustrative embodiments exemplifying the best mode of carrying out the disclosure as presently perceived.

One example of the present disclosure is a wearable multi-sensor device for measuring physiological properties. The example device includes at least one sensor housing including a plurality of non-invasive sensors including: a single-sided electrocardiography sensor, a bioimpedance and electrodermal activity sensor, and a photoplethysmography sensor. The device further includes a fixation element configured to secure a skin-facing side of the at least one sensor housing to exposed skin of a user and a communication module configured to receive signals from the plurality of non-invasive sensors and output data from the device. In some examples, the data is suitable for use in predicting glycemic events in the user. In some examples, the plurality of non-invasive sensors further includes one or more of a skin temperature sensor and a motion sensor, the motion sensor configured to sense motion of the user wearing the device. In some examples, the exposed skin of the user includes the user's upper arm. In some examples, the fixation element and at least one sensor housing are configured to locate the bioimpedance and electrodermal activity sensor proximal to a brachial artery location of the user. In some examples, bioimpedance and electrodermal activity sensor includes at least one current-sensing electrode and at least one voltage-sensing electrode. In some examples, the at least one current-sensing electrode and at least one voltage-sensing electrode are arranged to be placed in a line along a path of the brachial artery location. In some examples, the photoplethysmography sensor includes a multi-wavelength LED array. In some examples, the bioimpedance and electrodermal activity sensor includes a plurality of ECG electrodes. In some examples, the at least one sensor housing includes a plurality of ECG electrode housings each carrying one of the plurality of ECG electrodes, the plurality of ECG electrode housings being spaced apart from each other.

Another example of the present disclosure is aa method of predicting glycemic events in a user. The method includes, given at least one device worn by the user, the device including a plurality of non-invasive sensors positioned against skin of the user, the sensors including a single-sided electrocardiography sensor, a skin temperature sensor, a bioimpedance and electrodermal activity sensor, and a photoplethysmography sensor; collecting physiological signals of the user from the plurality of non-invasive sensors, generating data based on the physiological signals, the data being suitable for use in predicting glycemic events in the user, and processing the data using a prediction model executed by a computer processor, the prediction model returning, as an output, an indication of an occurrence of a glycemic event or a likelihood of occurrence of a glycemic event of the user.

In some examples, the method further includes providing the indication to the user;

In some examples, the method further includes transmitting the data from the device to a second electronic device, the second electronic device conducting the processing of the data. In some examples, the method further includes, before the processing of the data, conditioning the data and extracting a plurality of features from the data, the prediction model using the extracted features as an input to determine the indication. In some examples, the method further includes, before the processing of the data, identifying one or more salient features from the plurality of features and identifying correlations between the one or more salient features, the prediction model using the identified one or more salient features and the identified correlations as an input to determine the indication. In some examples, the method further includes, before the processing of the data, determining a suitable prediction model based on the one or more salient features. In some examples, extracting a plurality of features from the data includes extracting at least one of: heart rate variability measures, ECG, PPG, and BI beat morphology information, time-domain and frequency-domain from accelerometer and gyroscope data, skin temperature, or breathing waveforms. In some examples, the plurality of features extracted is selected based on their potential salience in predicting a future hypoglycemic and/or hyperglycemic event based on a score that ranks their importance and potential. In some examples, the indication is based on a predicted probability of a hypoglycemic and/or a hyperglycemic event calculated by the prediction model. In some examples, the prediction model is configured to use calculate beat level hypoglycemia and Hyperglycemia predictions.

In some examples, the method further includes extracting beat morphology features from the physiological signals, and where the prediction model is configured to determine the indication as a function of the extracted morphology features.

### BRIEF DESCRIPTION OF DRAWINGS

This disclosure will be more fully understood from the following detailed description taken in conjunction with the accompanying drawings, in which:

FIG. 1A is an illustration of one exemplary embodiment of an wearable multi-sensor device;

FIG. 1B is an illustration of another exemplary embodiment of an wearable multi-sensor device;

FIG. 2 is an isometric view of an example of a wearable multi-sensor device embodiment;

FIGS. 3A and 3B are front and back views, respectively, of a wearable multi-sensor device embodiment with strap-based sensors;

FIG. 3C is an illustration of the device of FIG. 3A in an installed configuration around an upper arm;

FIGS. 4A and 4B are front and back views, respectively, of a wearable multi-sensor device embodiment with patch-based sensors;

FIG. 5A is a schematic illustration of a control board and associated electrical components of the wearable multi-sensor device of FIG. 4A;

FIG. 5B is a schematic illustration of a sensor interface board and associated electrical components of the wearable multi-sensor device of FIG. 4A;

FIG. 6A is a front view of the wearable multi-sensor device embodiment of FIG. 4A with an adhesive patch;

FIGS. 6B and 6C are front and back views, respectively, of an arm with the wearable multi-sensor device embodiment of 4A in an installed configuration around an upper portion of the arm;

FIG. 7 is a photograph of an example wearable multi-sensor device;

FIG. 8 is a flowchart of a software protocol to predict hypoglycemia and hyperglycemia;

FIG. 9 is a schematic illustration of a dual-modal software protocol to predict hypoglycemia and hyperglycemia;

FIG. 10 is a block diagram of one exemplary embodiment of a circuit system for use within the devices of the present disclosure; and

FIG. 11 is a schematic illustration of cloud-based computing network for executing aspects of the present disclosure.

### DETAILED DESCRIPTION

Certain exemplary embodiments will now be described to provide an overall understanding of the principles of the structure, function, and use of the devices, systems, and methods disclosed herein. One or more examples of these embodiments are illustrated in the accompanying drawings. Those skilled in the art will understand that the devices, systems, components related to or otherwise part of such devices, systems, and methods specifically described herein and illustrated in the accompanying drawings are non-limiting embodiments and that the scope of the present disclosure is defined solely by the claims. The features illustrated or described in connection with one embodiment may be combined with the features of other embodiments. Such modifications and variations are intended to be included within the scope of the present disclosure. Some of the embodiments provided for herein may be schematic drawings, including possibly some that are not labeled as such but will be understood by a person skilled in the art to be schematic in nature. They may not be to scale or may be somewhat crude renderings of the disclosed components. A person skilled in the art will understand how to implement these teachings and incorporate them into work systems, methods, and components related to each of the same, provided for herein.

To the extent the present disclosure includes various terms for components and/or processes of the disclosed devices, systems, methods, and the like, one skilled in the art, in view of the claims, present disclosure, and knowledge of the skilled person, will understand such terms are merely examples of such components and/or processes, and other components, designs, processes, and/or actions are possible. In the present disclosure, like-numbered and like-lettered components of various embodiments generally have similar features when those components are of a similar nature and/or serve a similar purpose. To the extent terms such as front, back, top, bottom, proximal, distal, etc. are used to describe a location of various components of the various disclosures, such usage is by no means limiting, and is often used for convenience when describing various possible configurations. The foregoing notwithstanding, a person skilled in the art will recognize the common vernacular used with respect to medical devices and will give terms of those nature their commonly understood meaning.

To overcome the limitations of the current standard for glucose monitoring which includes invasive glucose monitoring techniques, examples of the present disclosure include fully noninvasive wearable to monitors and algorithms to predict, and alert in advance when a user is in a state of hypoglycemia or hyperglycemia. Example multi-sensor wearable devices can be positioned on the upper arm and examples can contain integrated sensors and accompanying circuitry to measure photoplethysmography (PPG), bioimpedance (BioZ), single-sided electrocardiogram (SS-ECG), electrodermal activity (EDA), and temperature. Accompanying the five sensors can be an accelerometer and gyroscope to provide contextual awareness as to what the wearer is doing (e.g., exercising). The accelerometer and gyroscope are also present to help mitigate noise due to macro-motion artifacts from the collected biosignals.

As an introduction to this example set of sensors, PPG measures a change in the blood volume of the probed tissues, BioZ measures a change in impedance due to changes in cellular shapes and orientations, which occurs with a pulse propagating through the blood vessels, SS-ECG measures an electrical biopotential from the heart, which occurs with every beat of the heart. These three modalities cannot only be used for predicting glycemic events, but for various cardiovascular diseases like hypertension via calculation of heart rate, heart rate variability, respiration rate, and potentially cuffless blood pressure. In addition, EDA is a measure of skin conductance and temperature is a measure of skin temperature fluctuations, which not only can contribute to the prediction of glycemic events but also can be used to determine emotional state or physiological stimulus such as stress. The accelerometer and gyroscope can be used to determine the position of the device relative to the user and provide contextual awareness to determine if the user is in a state of motion or is sedentary. All sensors combined in a single upper armband overcome the need for multiple noninvasive techniques to be positioned on the body such as the chest, wrist, or finger.

Thus, although one aspects of the present disclosure is the integration of the sensors and data in a multi-modal approach with unique algorithms to allow for a noninvasive means to predict hypoglycemic and hyperglycemic events before they occur, example system with alternative algorithms can be used for monitoring cardiovascular diseases like hypertension or for predicting emotional state or stress. The redundancy of the different modalities and features of each biosignal can also be used to mitigate noise such as motion artifact.

### Non-Invasive Multi-Modal Wearable Sensing Devices

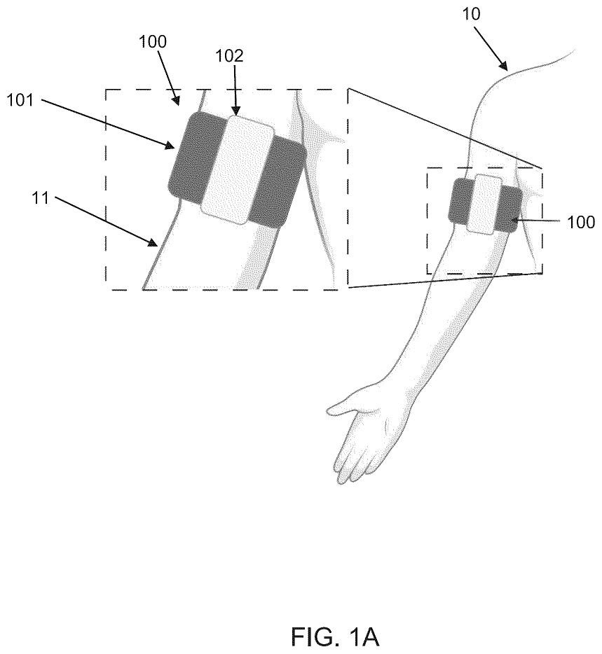

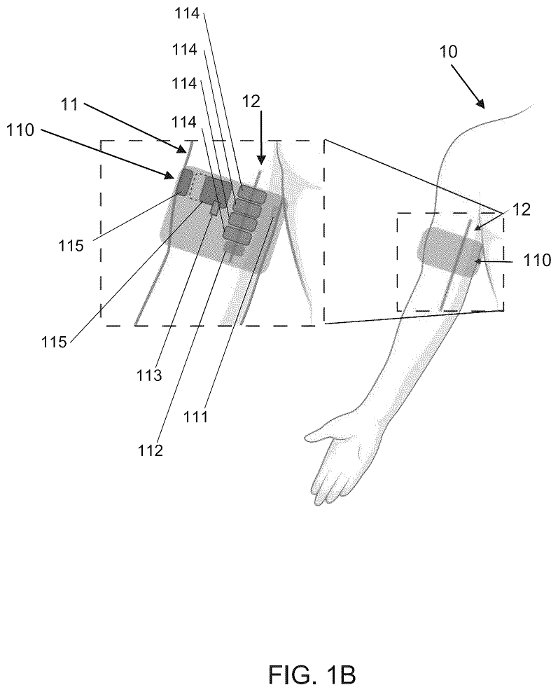

The focus of the present disclosures is described with respect to a multimodal sensing system in a wearable, unobtrusive, form factor with multiple biosensors. FIG. 1A shows an example device 100 placed on the upper arm 11 of a person 10 and containing an elastic, adjustable strap 101 and an electronic housing 102 for the microcontroller, circuitry, battery, and other components. FIG. 1B shows another device 110 with relative locations of all the sensors need to measure single-sided electrocardiography electrodes 115, bioimpedance and electrodermal electrodes 114, photoplethysmography sensor 112, temperature sensor 111, and accelerometer 113. FIG. 1B also shows a representative alignment of the bioimpedance and electrodermal electrodes 114 and photoplethysmography sensor 112 with a brachial artery path 12. All of sensors except the accelerometer can interface directly with the skin of the arm 11 and can be disposed on an inner surface of the device 110. The accelerometer can be housed with the rest of the supporting electronics inside a housing of the device 110.

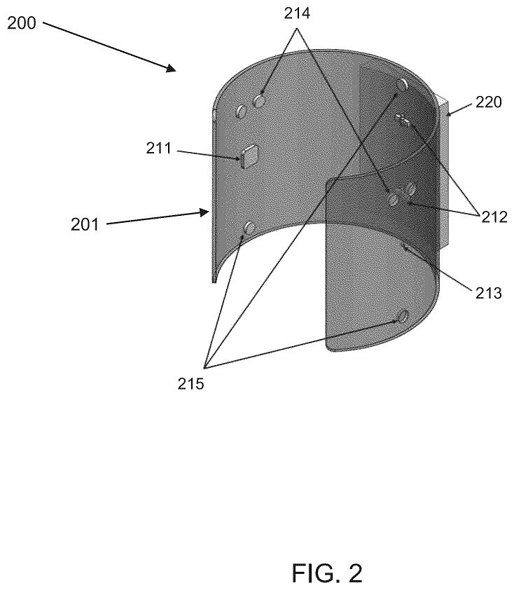

The device 200 of FIG. 2 illustrates one example of a semi-flexible arm band with a plurality of sensors, with the initial locations of the sensors, battery, microcontroller, and storage. In some examples, such as the device 200 of FIG. 2, six sensors are present: single-side electrocardiography sensors 215 (SS-ECG), bioimpedance (BI) and electrodermal activity (EDA) sensors (which can share the same electrodes 214), a temperature 211, photoplethysmography (PPG) sensors 212, and a motion sensor 214 (ACC) (e.g., accelerometer and/or gyroscope), with four electrodes 214 providing both the BI signal for HR/HRV and the EDA signal. Examples include wearable devices with sensors located to take advantage of these unique physiological biomarkers as well as the integration of the hardware and software. Examples include devices with multiple sensors to take advantage of the built-in redundancies to minimize noise (as described in more detail herein), and fit into a comfortable device (e.g., armband) to overcome some of the barriers associated with accuracy, sensitivity, robustness and usability. In some examples, sensed data is transmitted wirelessly to a smartphone or other electronic device so that the data can be used with a model to predict glycemic events, as described in more detail herein. Accordingly, the device 200 can include a housing 220 which can contains processing and control electronics, a display, and wireless communication transmitters.

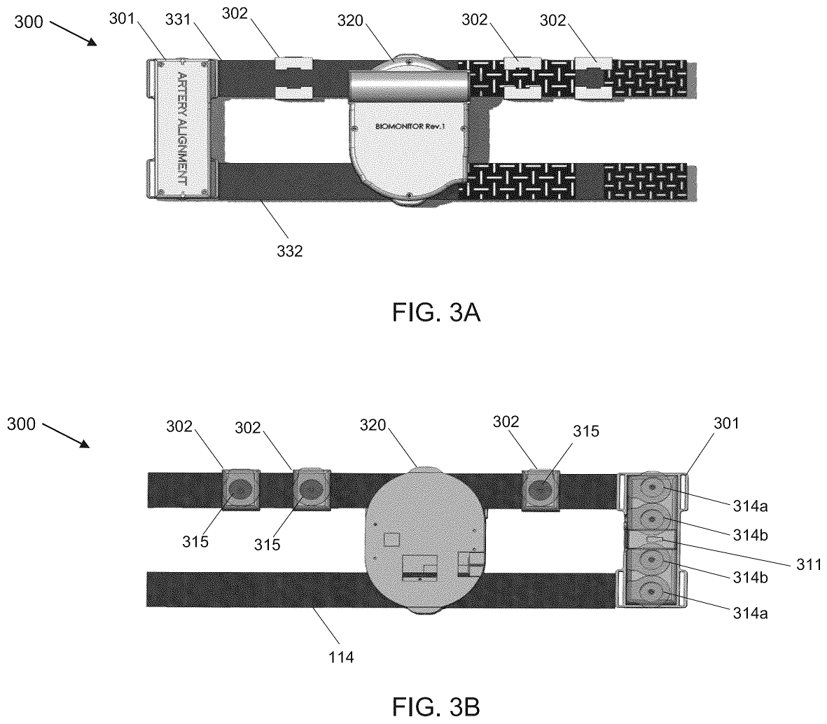

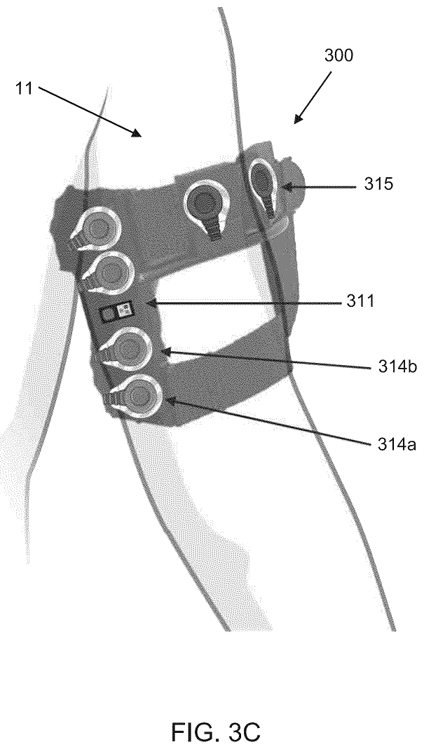

FIGS. 3A and 3B show outside and inside (e.g., skin-facing) views, respectively, of another example device 300 configured to be worn around the upper arm. The device 300 includes an arterial sensor housing 301, a circuitry housing 320 with an upper strap 331 and a lower strap 332 coupling the housings 301, 320 together. The upper strap 331 also carries three ECG sensor housings 302, each of which carries an ECG sensor electrode 315 on an arm-facing side of the sensor housing 302. The arterial sensor housing 301 includes, on a skin-facing side, as shown in FIG. 3, four BI/EDA electrodes (e.g., two current electrodes 314a and two voltage electrodes 314b arranged in two pairs), and sensor location 311 of a skin-contacting PPG sensor and a temperature sensor. The circuity housing 320 can include various circuity, including a BLE module, power management, ambient temperature sensor, a battery, and the accelerometer/gyroscope, for example. In operation, the straps 331, 332 are wrapped around an upper arm to secure the ECG sensor electrodes 315 and the BI/EDA electrodes 314a, 314b against the skin, with the BI/EDA electrodes 314 being aligned with the brachial artery of the arm. FIG. 3C shows the device 300 installed on an upper arm location.

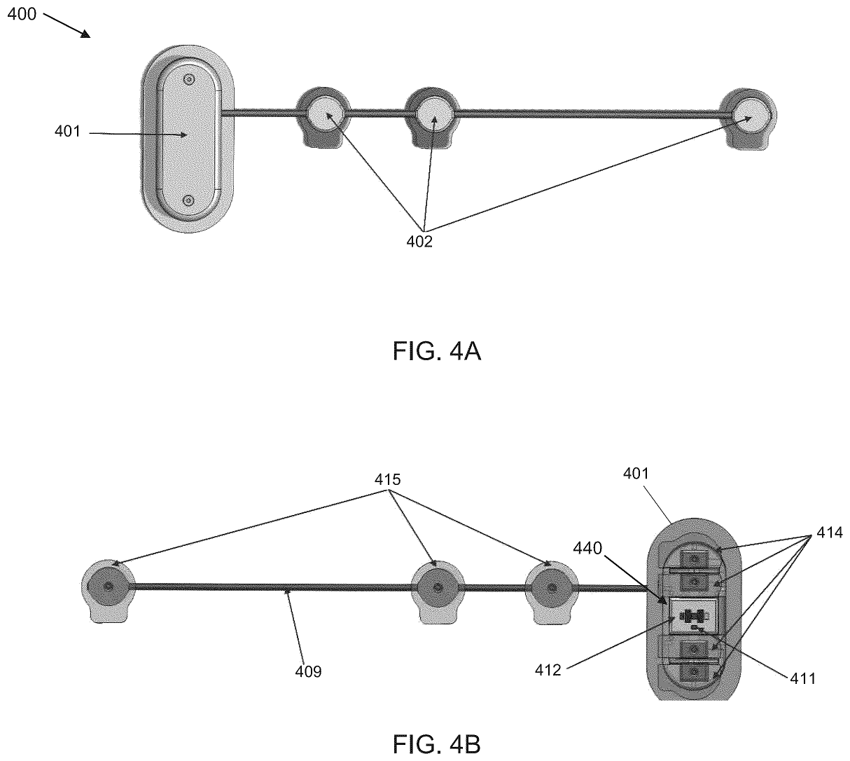

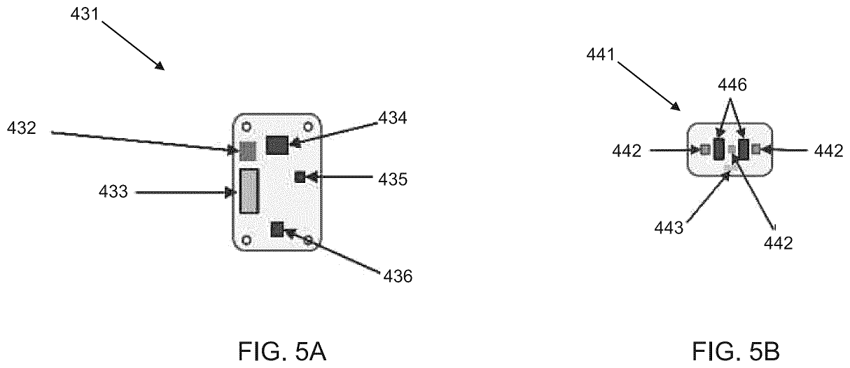

FIGS. 4A and 4B show outside and inside (e.g., skin-facing) views, respectively, of yet another example device 400 configured to be worn around the upper arm. The devices 400 includes a patch housing 401 configured to be disposed against the skin of an upper arm along the path of a brachial artery. A cable 409 extends from the patch housing 401 and includes three ECG sensor housings 402, each of which carries an ECG sensor patch electrode 415 on an arm-facing side of the sensor housing 402. The patch housing 401 includes, on the skin-facing side (as shown in FIG. 4B), a skin temperature sensor 411 a multiwavelength PPG sensor 412, and two pairs of BI/EDA electrodes 414. FIGS. 5A and 5B show example electronic components contained within the patch housing 401 of the device 400. FIG. 5A illustrates an example control board 431, with a wireless (e.g.,

Bluetooth) transmitter 432, a power management module 433, a microcontroller 434, a motion sensor 435 (e.g., accelerometer and/or gyroscope), and an ambient temperature sensor 436. FIG. 5B illustrates an example contract interface board 441 disposed within a skin-facing sensor housing 440 on the patch housing 401. The contract interface board 441 includes a multiwavelngth LED array 442 (e.g., green, red, IR), a skin temperature sensor 443, a photodiodes 446. The photodiodes 446 and LED array 443 can operate together as the PPG sensor 412.

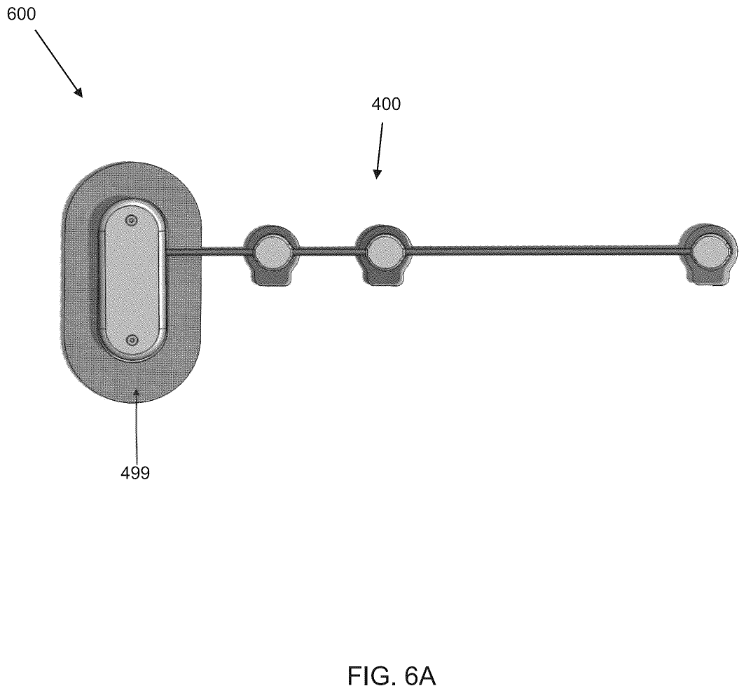

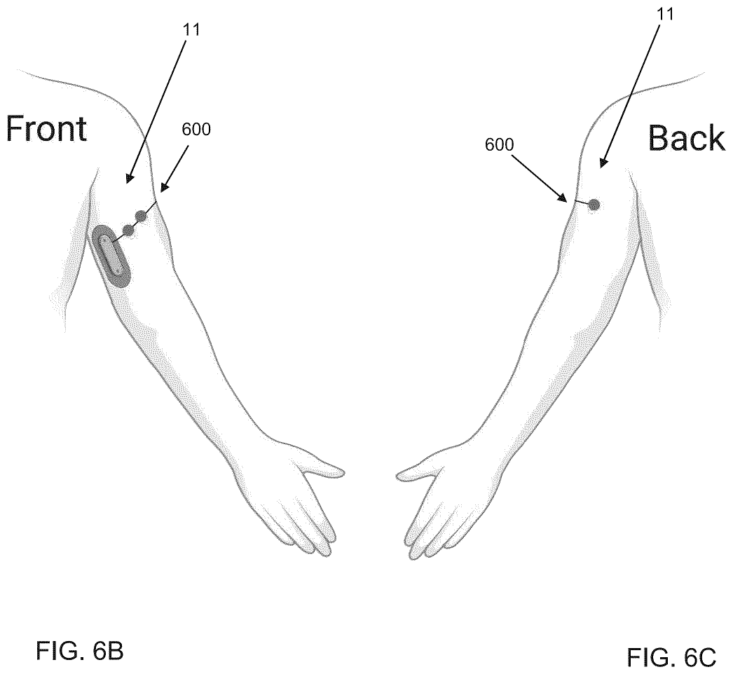

In operation, the device 400 can be used with via an adhesive patch 499, as shown in FIG. 6A, that is configured to secure the patch housing 401 to the skin of the arm, as shown in FIGS. 6B and 6C, with the patch housing 401 disposed against the skin of an upper arm 11 along the path of a brachial artery.

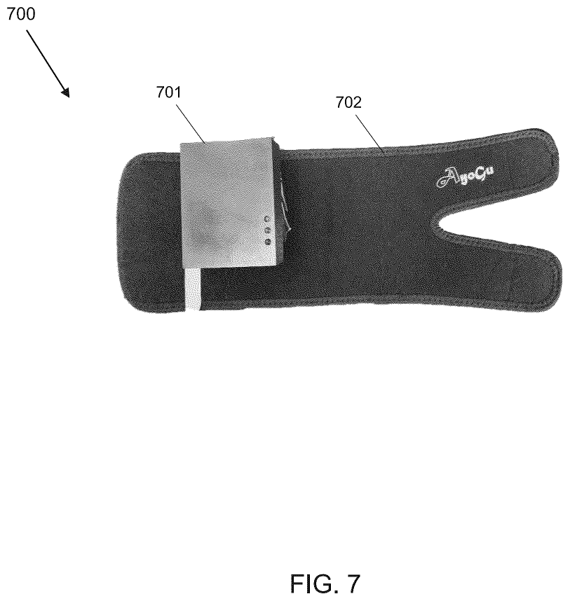

FIG. 7 is a photograph of an example device 700 that includes a housing 701 and an armband 702 for securing the housing against an upper arm location. The housing 701 can include any of the sensors and electronics components disclosed herein, including a plurality of electrodes and skin-contacting sensors arranged to contact the skin of the arm when the armband is holding the housing 701 against the arm. Additionally, while many of the devices disclosed herein are configured for use around the upper arm, other locations are suitable, such as around the lower arm, other location or other appendage, as well as on other parts of the body. Additionally, while the examples illustrated herein have their sensors associated with a single device, other implementations are considered, such as two or more devices with separate locations on the body. For example, an example device and sensor suite, if can be configured for use on the chest (e.g., across the heart), and can provide more features from the actual traditional ECG signal than is provided from the single-sided-ECG. Other unique features on the chest can reveal additional cardiac features due to the proximity of the sensor suite to the heart, where time domain separation features need not be considered, allowing for instantaneous signal alignment where using reference diagrams, such as the Wiggers diagram, would be useful in evaluating baseline drifts associated with hypo/hyper glycemic events Beyond glycemia, placing such a sensor suite at multiple locations can provide features for monitoring cardiac relevant signals such as cuffless blood pressure or, if in two locations, peripheral artery disease by getting the Ankle Brachial Index by dividing the blood pressure, or equivalent signals from the sensors, located in an artery of the ankle by the blood pressure, or equivalent signal from these sensors, located in the artery of the arm. Additionally, the sensor suite can be located in the upper portion of the leg, giving access to the popliteal and anterior tibial artery, allowing the device to monitor pulse pressure response, which is useful with individuals who have had amputations.

Some of the sensed signals (e.g., ECG signals) are vulnerable to distortions caused by motion artifacts. Motion artifacts can come from several sources, but particularly from macro-motions due to physical activity and micro-motions due to breathing or light sensor movements relative to the heterogeneous tissue. These artifacts can be reduced for heartrate estimation by averaging a signal over tens of seconds, but this challenges the ability to get HRV. To overcome the macro-and micro-motion artifact noise inherent in the signals and provide robust signals, examples of the present disclosure can acquire both ACC data for macro-motion and three redundant signals (e.g., SS-ECG, BI and PPG) that will contain the HR/HRV signals and macro/micro motion noise. The ACC signal can help to eliminate macro-motion while also providing contextual information regarding what the patient is doing (e.g., exercise), which can be used to assist in the prediction of glycemic events. The other three signals (e.g., SS-ECG, BI and PPG) can each provide a measure of HR and HRV; however, they all come from different physiologic phenomena, which provides several advantages. In particular, the SS-ECG is effectively the electrical signal from the heart, and as such, can provide a narrower signal than PPG or BI. Thus, SS-ECG can be used as the primary source of information for generating HR and HRV. However, since SS-ECG signal can be single sided, a full ECG waveform may not be available for use in predictive models, as discussed herein. The BI and PPG signals are from different physiologic sources (e.g., impedance changes with changing blood flow and optical absorption changes with changing blood volume in the arteries and arterioles), the entire set of features from their waveforms (e.g., wave shape, dicrotic notch, etc.) can be used. In addition, PPG and BI can provide redundancy in HR/HRV measurement that can generate more robust and accurate signals by eliminating the common mode noise (e.g., from motion artifacts).

In particular, to get a strong physiological signal for biosensing in the upper arm, examples can include Ag/AgCl electrodes with an ultra-low power ECG analog front-end, which can provide a good signal-to noise for the SS-ECG detections. The PPG sensors 212 can include multiple LED wavelengths to enable differentiation of the signal in the presence of different skin tones, from dark to light, as per the Fitzpatrick scale and thereby mitigate another common noise factor with these systems for diverse populations. Thus, the PPG sensor system 212 can include two sensor packs placed in two locations on the upper arm to ensure good contact and signal integrity. The sensor packs can consist of three LEDs (e.g., red, green, and infrared) with two photodiode detectors placed (e.g., ˜1 cm) from the center point of the LEDs. Examples utilizing three sensors are able to mitigate the skin tone variation and have redundancy built within this modality as all three wavelengths can be measured separately and will contain common mode noise. In some examples, the temperature sensor 211 is a contact sensor placed on the inside of the arm near the armpit of the user to accurately measure the axillary temperature. The ACC sensor 213 can be placed opposite the temperature 211 sensor on the outside of the arm (as shown in FIG. 2) to capture macro motions. Example BI-EDA sensor arrangement include the use of four Ag/AgCl electrodes 214 placed on opposite sides of the upper arm (as shown in FIG. 2), two for injection and two for receiving the signal. Additionally, because resting HR can vary between 60-100 bpm (1-1.7 bps) and in extreme cases can go to 30 bpm (0.5 bps) or above 400 bpm (6.7 bps) and because normal HRV can range from above 100 ms between beats for healthy subjects to below 50 ms between beats for unhealthy subjects, the three HR and HRV sensors can be sampled at least within 1 ms.

The devices disclosed herein can include printed circuit boards (PCB), which can have a rigid-flex design so that the multimodal sensors make sufficient skin contact via flexible circuit design while maintaining signal integrity and structure. An example PCB can include four main components: (1) wireless microcontroller to collect and distribute sensors signals, (2) onboard non-volatile storage, (3) signal processor(s), and (4) a battery.

For the integration of these components into a single form factor examples can include using ultra-low power sensors, transducers and analog front-end (AFE) for sensors. Examples can include optical, electrical, temperature and acceleration sensors. The sensors can be activated with a relatively low duty cycle (e.g., ˜12 times every hour for less than 1 minute). Typical power budgets for these sensors that include LEDs (e.g., ˜1 mW), trans-impedance amplifiers (e.g., 10-100 uW), ADCs (e.g., 10-100 uW), and microcontrollers (e.g., 100 uW-1 mW) are in the range of 5-10 mW. Considering the activation cycle for these sensors, the total power consumption will be in the order mW. Electrical sensors that measure bio-potentials or perform impedance spectroscopy present potentially less complexity as optical actuation is not required. Some examples include a total power budget of 4-5 mW for instrumentation amplifiers, ADCs and microcontrollers. Accordingly, example devices are expected to remain operational for one full day on a 250 mAh rechargeable battery (e.g., with a volume of less than 2.75 cm³ and a weight of about 6 g) without the need for recharging. Example devices include the use of reduced PPG wavelengths (e.g., if not required for accommodating things like variation in skin tone). In some examples, PPG sensors are absent and only BI and SS-ECG are used such as when you might have deeper vessels to far for the light from the PPG to reach the vessels or when you have darker skin tones, causing the light for the PPG signal not to penetrate the tissue as well. Examples of the present disclosure also include devices with an SS-ECG sensor placed closer to the shoulder (e.g., a single lead to a shoulder patch).

Once data is collected, it can be helpful to subject it to processing to, for example, filter out noise. For example, raw ECG signals can be processed in multiple steps to extract HRV features. A first step can include the ECG signal being passed through a high-pass filter (e.g., above 0.5 Hz) to remove baseline wander. In a second step, a continuous wavelet transform (CWT) can be applied with a Ricker wavelet at a center frequency (e.g. 0.25 Hz) to attenuate auxiliary peaks and make the R-peaks more prominent. The two steps make it easier to detect R peaks in the ECG signal.

### Data Collection and Processing Examples

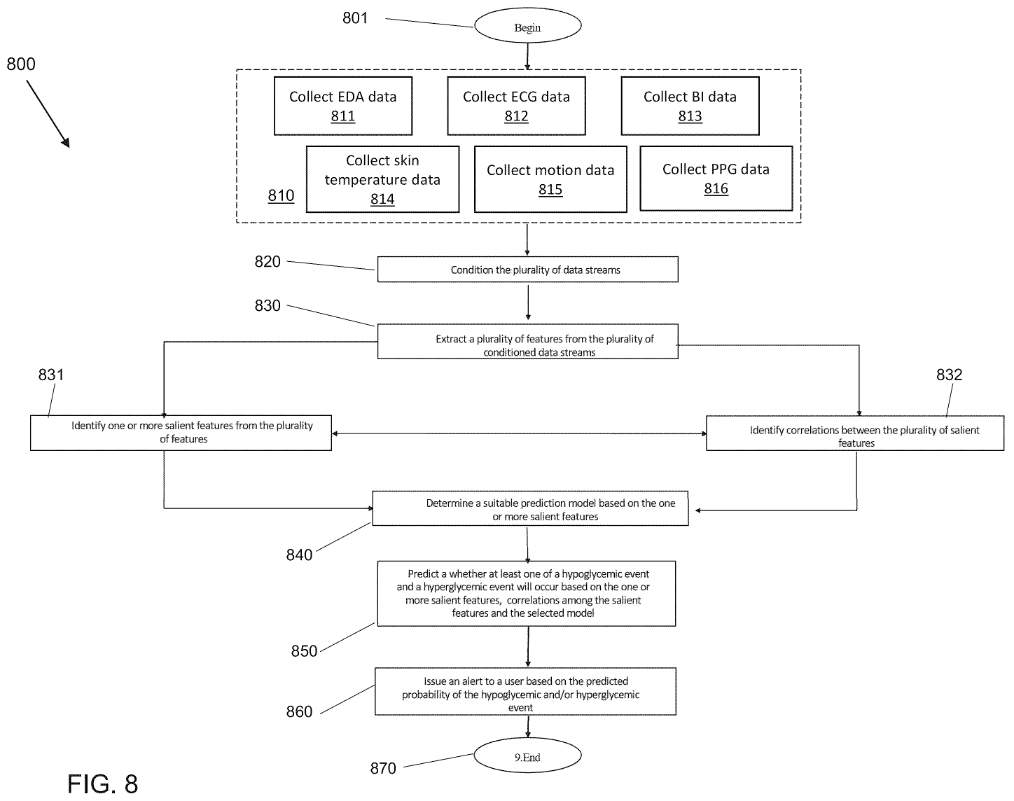

FIG. 8 is a flow chart of an example computer-implemented protocol to predict hypoglycemia and hyperglycemia using data from, for example, the multi-modal non-invasive wearable devices of the present disclosure. The example process 800 begins with a data collection 810 of a plurality of data streams. The data streams collected may include data from each of the sensors of the devices, or example data collection 812 from a single-sided electrocardiogram (SS-ECG), data collection 816 from a photoplethysmography (PPG) sensor, data collection 813 from a bioimpedance (BioZ) sensor, data collection 814 from a skin temperature sensor, data collection 811 from an electrodermal activity (EDA) sensor, a data collection 815 from motion sensors, including accelerometry, and gyroscope data streams. The data streams can be collected from one or more users at periodic intervals in a non-invasive manner.

Once the data is collected from a patient, the data streams collected are conditioned 820. This conditioning 820 step can involve integrating, aligning, and/or cleaning the captured data. For example, erroneous data can be removed based on determining filtering criteria. As a second example, a first data stream can be integrated with a second data stream, a third data stream, etc., to mitigate or remove common mode noise such as motion artifacts. For example, the PPG, BioZ, and SS-ECG data stream can each be integrated to eliminate noise in a heart rate or respiration rate calculation. Further, the conditioning 820 can involve converting timestamps to a standard time zone based on different time zones. This can be useful to align a timestamped first data stream with a timestamped second data stream such that the timestamps correspond between the two data streams. For an example, the reference signal CGM measurements can be collected at a periodic interval (e.g., every five minutes).

Each CGM measurement can be mapped to the nearest minute to remove any minor variations between each period.

Next, a plurality of features is extracted 830 or determined from the plurality of conditioned data streams. At least some of the features extracted can be derived from one or more of the conditioned data streams but may not comprise raw data and thus can be unique from any of the raw data collected from the patient. Features extracted from one or more raw data streams can comprise heart rate variability measures, ECG beat morphology information, time-domain and frequency-domain from accelerometer data, skin temperature, and breathing waveforms. The plurality of features extracted can be selected based on their potential salience in predicting a future hypoglycemic and/or hyperglycemic event based on a score that ranks their importance and potential.

Next, examples include identifying 831 one or more salient features from the plurality of extracted features. This can be done via the use of machine learning algorithms (e.g., random forests) that determine the importance of the feature based on its impact on the prediction performance. Another way is to evaluate the impact of the extracted salient features is by combining them as input to deep learning methodologies. The ideal set of features can be determined based on the exhaustive combinations of features used and its impact on the prediction performance. The process can also include identifying 832 the correlation between the pluralities of extracted features. As an example, features extracted from ECG data and accelerometer-based features from another device. This can be used in determining the quality of the data and/or provide information on the usefulness of data streams being collected. The correlation metrics can be used for a selection of one or more features that adds value to the prediction of hypoglycemia and/or hyperglycemia. With the salient features and their correlations identified, a suitable predict model is selected 840.

Next, examples include the use of machine learning and deep learning for using the plurality of extracted features to predict 850 hypoglycemia and/or hyperglycemia events. This can involve using separate traditional machine learning-based algorithms like random forests and more sophisticated deep learning algorithms using convolutional neural network (CNN) layers, long short-term memory (LSTM), and fully connected to determine the prediction. Example method can be extended to combine the prediction power of both these approaches in overall improved performance. The predicting 850 can include determining whether or not at least one of a hypoglycemic event and a hyperglycemic event will occur based on the one or more salient features and the selected prediction model selected. This step can involve using an optimized prediction model to predict a probabilistic estimate of hypoglycemic vs. hyperglycemic event. The threshold probability of at least one hypoglycemic event and a hyperglycemic event required to issue a positive prediction can be, for example, a probability equal to or greater than 50%; however, the threshold probability can vary in other embodiments. Additionally, the threshold probability can be a function of one or more features extracted or user requirements. For example, the threshold probability required to issue a positive prediction can be reduced if the user is currently engaged in a hazardous activity, such as driving or during sleep, given that the consequences of a hypoglycemic/hyperglycemic event occurring during the performance of a hazardous activity can be elevated

After the prediction, an alert can be issued 860 to a user or other party (e.g., caretaker, nurse, doctor, etc.) based on the probability of at least one of hypoglycemic and a hyperglycemic event predicted. The alert can be communicated to and issued from a portable computing device in proximity to the user, such as a smartphone or other device worn by the user. The alert can also be issued through computing devices connected to the portable computing device, such as through the speakers of a vehicle connected to the portable device through short-wave radio signals. The alert can be configured to obtain the user's attention, whereby the user can perform a corrective action to address the predicted hypoglycemic/hyperglycemic event, such as through the administration of insulin or carbohydrate intake.

The combination of multiple modalities, provide the ability to, in some examples, cross-correlate the measurements and determine accurately when a subject is going to be hypoglycemic or hyperglycemic. The multiple biosensors also provides noise reduction and contextual awareness for when the subject would be most likely to have an event while also providing determination through other physiological monitoring that a subject is going to have an event.

Beat level hypoglycemia and Hyperglycemia predictions can be based on morphology features extracted from ECG, PPG, and EDA sensor data obtained from, for example, the devices disclosed herein. Morphology features that are significant for beat level predictions are can be identified. Examples of the present disclosure include the use of machine-learning and deep-leaning models to predict beat level hypoglycemia and hyperglycemia based on the fused significant morphology features.

In some examples, beat level hypoglycemia and hyperglycemia predictions are fused with contextual information such as skin temperature data and accelerometer data (e.g., from a wearable device example of the present disclosure). The contextual information can provide information on physical activity and perspiration. Beat level predictions can then be fused with contextual information to refine the beat level predictions. This modular (e.g., stacked) prediction refinement approach enables accommodation of predictions for different combinations of sensor availability.

Examples of the present disclosure include aggregation of beat level predictions to time window based hypoglycemia and hyperglycemia predictions. In some examples, individual beat level predictions are aggregated into a time window (e.g., 5 minutes) prediction using percentage and sequence of beat level predictions. Examples include the use of machine-learning and deep-learning models, along with rule-based patterns to aggregate to time windows based predictions. Examples of the present disclosure include generating time window based hypo and hyperglycemia predictions, which can provide clinically relevant predictions.

### Systems and Methods for Predicting Glycemic Events

The ability to predict future glucose levels based on past measurements can lead to a number of therapeutic solutions, ranging from the ability to warn patients of future hypoglycemic events to delivering insulin automatically. Predictive models can be classified into two broad categories: physiological models and data-driven models. Physiological models typically rely on ordinary differential equations (ODE) models of glucose metabolism and insulin action. Physiological models are able to simulate detailed glucose and insulin responses to carbohydrate intake, and in some cases fat/protein consumption and exercise events. In contrast, data-driven (or empirical) approaches “learn” a prediction model directly from data (e.g., from CGMs). A vast pool of machine-learning techniques can be used for this purpose, including autoregressive (AR) models, neural networks, random forests, Kalman filters and support vector regression (SVR). These models can be trained to predict the actual glucose level at a future time (e.g., a regression problem) or to generate hypoglycemia/hyperglycemia alerts (e.g., a classification problem). Following trends in machine learning, recent research in glucose forecasting has focused on deep-learning techniques. However, existing machine-learning studies attempt to predict future glucose values from past glucose measurements (e.g., using CGMs), which is fundamentally different from predicting hypoglycemic events. Accordingly, there exist a need for a system to predict hyperglycemia from non-invasive measurements.

Examples of the present disclosure include machine-learning methodologies that combines information from the multiple non-invasive physiological sensors of Aim #1 to predict glycemic events. For examples, two types of models are considered that can predict hypoglycemic events and clinical indicators of glucose control (e.g., hyperglycemic time-in-range). Some examples include using non-invasive physiological sensors to replace direct glucose measurements (e.g., from CGMs), but other examples include situations with direct sensors used to augment CGM devices to improve glucose predictions. Examples also include prediction scenarios with various time horizons 0<H<60. These prediction models follow similar design, development, test, and performance evaluation processes. Example model developments are described below with model-specific details highlighted when appropriate.

Examples include the use of two complementary methodologies to build these models, based on statistical learning (SL) techniques and deep learning (DL). The boundaries between machine learning, statistical learning and deep learning are blurry. Here, the term statistical learning is used to refer to traditional techniques (e.g., nearest neighbors, decision trees, support vector machines), and deep learning to refer to techniques based on convolutional and recurrent neural networks. The SL methodology enables, for example, the use of a rich set of features from the multimodal data and develop predictive models, whereas the DL methodology enables extraction of information automatically from raw sensor signals (e.g., via convolutional networks) and integrate temporal information (e.g., via recurrent networks.) Example multimodal data include the following:

Demographic: gender, age, time since diabetes diagnosis, T1 or T2D, last HbAlc reading, historical diabetes management profile (average glucose, # hypoglycemic events, % time in-range and in-hyperglycemic-range)

Context (ACC): hour of the day, potential sleep and motion activities based on data collected, body posture, exercise level. For example, if HRV is low and skin temperature is high, a flag can be generated to indicate that exercise might be taking place. On the other hand, if both HRV and skin temperature are low, the patient might be experiencing a hypoglycemic event. Examples include inferring from summary statistics such as mean, standard deviation and energy

CGM: current glucose value, change in glucose over time (e.g., 5, 10 minutes); variance (e.g., in the last 30 and 60 mins); accumulated positive and negative changes (e.g., in the last 30 and 60 mins), glucose.

SS-ECG: Heart rate (HR), time domain HRV features (e.g., SDNN, pNN50), frequency domain HRV features (e.g., power in ULF, VLF, LF and HF bands) non-linear features (e.g., Poincare plots, entropy).

PPG and BI: HR, time-& frequency-domain HRV features, AC amplitude and DC offset.

SS-ECG+PPG+BI: pulse transit time, and potential for correlation with blood pressure.

Temperature: Axillary body temperature measurement.

EDA: Phasic skin conductance response (SCR−median EDA in the last 4 sec, for example); peak amplitude (e.g., in the last minute), rise time, number of SCR peaks (e.g., in the last 5 minutes); average SCR (e.g., in the last 5 minutes).

Examples include the development of the SL prediction models, which can be conducted in, for example, four phases: (1) Data curation: Data collected from different sensor streams is checked for missing data and outliers. For example, if data is missing or noisy in one data stream, corresponding temporally matched data from other data streams is eliminated from the analysis to ensure completeness and integrity of the integrated data. All the different data streams can be indexed by timestamp and integrated into a common schema for data analysis and model development. (2) Exploratory data analysis: Exploratory data analysis can be conducted on the integrated data set and to generate visualizations that analyze patterns of individual data streams as well as pairwise correlations. For example, correlation and deviance analysis between HRV derived from ECG, BI, and PPG can provide insights on reliability, fidelity, and how context (e.g., motion artifacts) affect these redundant data streams. Correlation plots between HRV and hyperglycemia and hypoglycemia provide insights on the nature of the relationship between these important features. Additionally, examples include characterizing patients based on demographics and historical diabetes profile. (3) Feature engineering: Glucose prediction techniques can be used to extract a rich set of features that capture the patterns in different data streams. For example, extracting features based on the current snapshot (e.g., features at a point in time), short-term features (e.g., changes and patterns in the last few minutes), medium term features (e.g., changes in the last 2 hours), and snowball features (e.g., changes that are accumulated over time). (4) Model development: Examples can include hypoglycemia prediction using Random Forests (RF), which is known to be very effective in 30 and 60 minute time horizon. Examples also include using other SL techniques (e.g., support vector machines, gradient boosting, Gaussian process regression), and dependent variables beyond hypoglycemia, such as predicting time-in-range.

Examples include developing prediction models using the complete set of available features and features that are significant for the model performance identified using p-values, variable importance plots, mutual information, LASSO, and minimum redundancy maximum relevance (mRMR). The goal of feature selection can be to identify a parsimonious combination of features with good performance results. Examples include determination of optimal combination of sensors required for predictive model development, low cost, low power, and high reliability. For example, examples include assess whether redundancy of ECG, BI, and PPG is required to extract HRV for good model performance under different motion and environmental contexts. Examples also include assessing whether features from these or the ACC, Temp or EDA sensor can be dropped without affecting performance to minimize complexity and save power (e.g., in some devices the PPG system is the biggest power consumer).

Deep learning has achieved remarkable success in image, video and speech processing, and this success have also permeated health related applications. Examples include detecting atrial fibrillation using smartwatches and performing medical imaging diagnoses that compete with human experts, to name a few. Part of this success is due to deep learning's ability to perform two challenging and complementary tasks: (1) extract relevant information automatically from raw data (e.g., without the need for the feature engineering step described above); and (2) integrate such information when it appears in a sequence. These two tasks are can be achieved through the use of convolutional neural networks (CNNs), and recurrent neural networks such as Short-Long-Term-Memories (LSTMs), respectively.

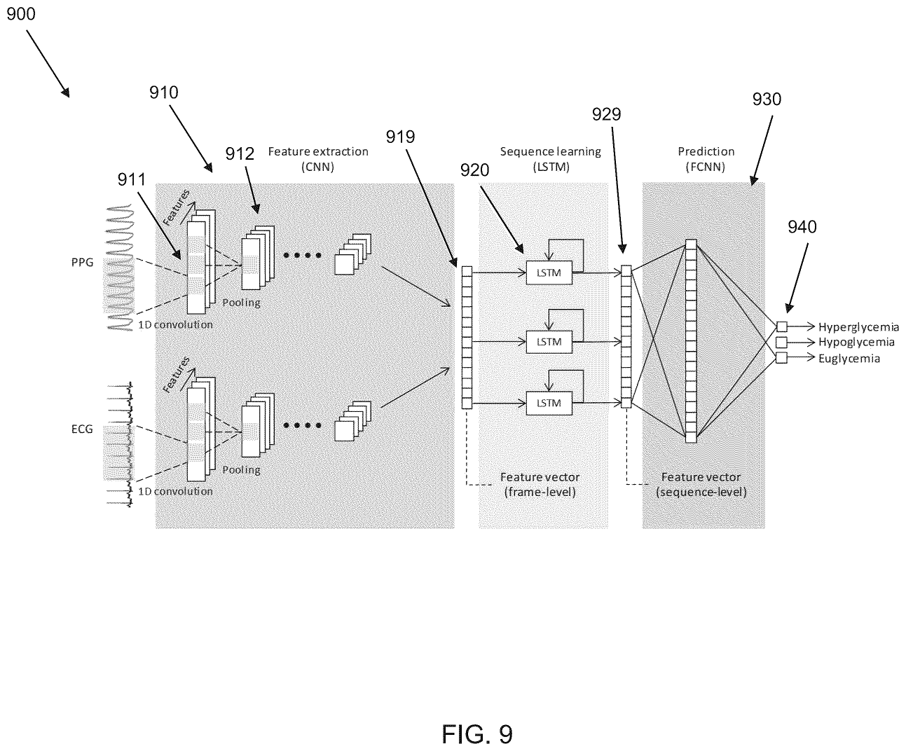

Examples of the present disclosure include the development, training, and use of deep learning architectures that incorporates both types of networks, as illustrated in FIG. 9, where for simplicity only ECG and PPG channels are illustrated. Short analysis windows or frames (e.g., 10 sec) for each channel are processed with a dedicated CNN 910. Each CNN 910 performs two basic operations: applying a convolution filter to each frame, and then subsampling the outputs (e.g., pooling). By repeating these two operation over multiple layers, the CNN 910 extracts increasingly more complex representations of the raw sensor signal. Outputs from the last layer of all the CNNs 910 are then concatenated to generate a feature vector 919 that captures information across all the input channels. As the analysis window moves over the sensor data stream, sequences of these frame-level feature vectors are then “digested” by a set of LSTMs 920, which then produces a feature vector 929 that summarizes information across time. The final sequence-level feature vector 929 is then processed with a fully connected neural network (FCNN) 930, which generates a prediction (e.g., upcoming hypoglycemic event).

To overcome the macro-and micro-motion artifact noise inherent in the signals and provide robust signals, we also acquire the accelerometer (ACC) and gyroscope (GYRO) data for macro-motion. Note that the feature extraction from the ACC and GYRO signals can both help to eliminate macro-motion and provide contextual information regarding what the patient is doing like exercise, which is useful in the algorithms to calculate glycemia. Further skin temperature and skin conductivity have features including a decrease in their values with the onset of hypoglycemia which could be used to enhance the algorithm. Glucose levels can potentially exhibit a nonlinear relationship with ECG and PPG signals due to external factors. Thus, additional multimodal data, including skin temperature and accelerometry, enables the capture of changes attributed to these external factors, which alone could be useful as well as in an algorithm with the other sensors toward facilitating a more direct relationship with glucose readings.

Examples include training deep-learning architectures using, for examples, datasets containing simultaneous recordings from multiple sensors, such as the MIMIC-II Waveform Dataset (e.g., ECG, PPG, respiration), the 2015 IEEE Signal Processing Cup Dataset (e.g., ECG, PPG, acceleration), the Vortal Dataset (e.g., ECG, PPG, respiration), the PPG-DaLiA dataset (e.g., ECG, PPG, respiration), and the CASE dataset (e.g., ECG, PPG, SC). Examples also include using datasets to pre-train each channel's CNN independently (e.g., a CNN for the PPG channel is pre-trained to predict HR and HRV). Once pre-trained, the CNNs are combined with the rest of the architecture (e.g., LSTM, FCNN) and trained jointly.

The performance of both types of machine-learning models (e.g., statistical, deep) can be assessed systematically using suitable measures for each dependent variable and can be compared with performance of CGM based models. Specifically, examples include evaluating example models trained to predict hypoglycemia using sensitivity, specificity, precision, recall, as well as ROC curves. In addition, examples include evaluating models trained to predict time-in-range for hyperglycemia using Pearson correlation and root mean squared error (RMSE). In addition to cross validation (CV) based performance assessment, which relies on random sampling of training and test datasets, examples of the present disclosure include: Partitioning data based on patients (e.g., model training performed on a subset of patients and testing performed on a different set of patients to promote generalizability and reproducibility for new patients). Partitioning data based on time (e.g., model training performed on a subset time period for each patient and testing done on the data from a different time period, such as a different day, to promote temporal generalizability and reproducibility of the model). And pooled data based validation (e.g., models generated from data collected from different phases are compared for model stability and a final model is developed on the combined data collected over all the phases using CV to avoid over fitting).

However, as mentioned before, motion artifacts present a potential risk for the model development, which are always an issue when working with wearable physiological sensors. However, the multisensory system examples disclosed herein provide sufficient redundancy (e.g., three independent measures of HR and HRV) and contextual information (e.g., accelerometer data) to overcome motion artifacts through signal processing.

Moreover, examples of the present disclosure can include a number of refinements to improve predictive performance. For example, while ECG beat morphology may outperform R-R time series marginally for hypoglycemia prediction, examples include combining the two sets of features (e.g., ECG beat morphology and R-R time series) to provide a statistically significant improvement. Examples include several strategies to further improve predictive accuracy, such as:

Threshold optimization: While hypoglycemia is generally defined as having glucose levels below 70 mg/dl, prior research indicates that physiological changes only become prominent at lower glucose levels (e.g., 50 mg/dl). In addition, commercial CGMs are less accurate at lower glucose levels. Examples include lowering the glucose threshold, which can improve the prediction of hypoglycemia.

Universal donors and receptors: Examples include the use of a validation protocol that includes a personalized model for each participant in the training set and then matches a test participant to the training participant whose model provides the best predictions. This gives rise to the notion of universal donors (e.g., training subjects whose models work well across multiple test subjects) and universal receptors (e.g., test subjects whose data works well across models from multiple training subjects).

ECG beat segmentation: While examples include extracting ECG beat morphology using a fixed-length window centered on the R peak, beat morphology is known to change with heart rate. As an example, an increase in heart rate (reduction of RR interval) leads to a reduction of the QT interval. As a result, a fixed analysis window runs the risk of including information from multiple heartbeats. To address this issue, examples include replacing the fixed analysis window with one whose length is proportional to the RR interval.

Class imbalance: A major issue in hypoglycemia prediction is class imbalance: for most subjects, hypoglycemic readings represent a small percentage (1-5%) of all CGM readings. Examples of the present disclosure include different approaches to address this issue, such as:

Data augmentation: Each CGM reading is associated with a time series of heartbeats (e.g., 5-minutes) and, rather than extract a single analysis window from this time series, examples include extracting multiple overlapping analysis windows and treat each as a new training example. Using this strategy on hypoglycemic CGM readings, examples can “oversample” the minority class and reduce the class imbalance. Results indicate that this strategy increases predictive accuracy by up to 5%.

Loss function: The conventional loss function to train a deep-learning classifier, known as the cross-entropy loss, does not account for class imbalance in the datasets. For this reason, examples include the use of additional loss functions, such as balanced cross-entropy. Results indicate that this new loss function can improve predictive accuracy by up to 5%.

Ensemble learning: While some example prediction models generate a separate prediction for each ECG beat or ECG beat-sequence, this may ignore the fact that glucose fluctuates at a much slower rate than the physiological signals. To address this issue, examples include the use of ensemble-learning methods to aggregate predictions from multiple ECG beats and beat-sequences associated with the same CGM reading.

Examples include different approaches to aggregate the individual beat-level predictions to an interval for better interpretability and predictive performance such as a fusion-based approach in which the machine learning (ML) model is trained to learn morphological changes occurring over a range of glucose thresholds instead of a specific threshold. For detecting morphological changes at 70 mg/dL, the changes occurring in the neighborhood of 70 are useful and including these additional features in the model enables better learning of morphological changes occurring in the low glycemic range (and high glycemic range in the case of hyperglycemia).

### Computer Systems and Cloud-Based Implementations

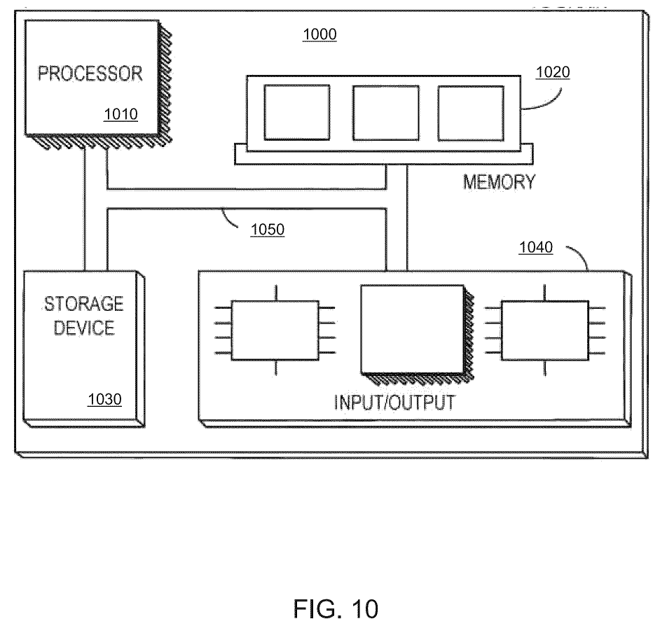

FIG. 10 is a block diagram of one exemplary embodiment of a computer system 1000 upon which the present disclosures can be built, performed, trained, etc. For example, referring to FIGS. 1A to 9, any modules or systems can be examples of the system 1000 described herein, for example any of the sensors and associated electronics as well as any of the associated modules or routines described herein (e.g., prediction algorithms). The system 1500 can include a processor 1010, a memory 1520, a storage device 1030, and an input/output device 1040. Each of the components 1010, 1500, 1530, and 1040 can be interconnected, for example, using a system bus 1050. The processor 1010 can be capable of processing instructions for execution within the system 1000. The processor 1510 can be a single-threaded processor, a multi-threaded processor, or similar device. The processor 1510 can be capable of processing instructions stored in the memory 1520 or on the storage device 1030. The processor 1010 can execute operations such as controlling sensor operation, including turning sensors on and off and receiving data from sensors, as well as executing logic related to senor timing, data transmission, and the prediction techniques disclosed herein and outputting a result, operating a display device of a computer system, such as a mobile device, to visually present from data any of the metrics and algorithms disclosed herein, among other features described in conjunction with the present disclosure.

The memory 1020 can store information within the system 1000. In some implementations, the memory 1020 can be a computer-readable medium. The memory 1020 can, for example, be a volatile memory unit or a non-volatile memory unit. In some implementations, the memory 1020 can store information related functions for executing objective audio processing metrics and any algorithms disclosed herein. The memory 1020 can also store digital audio data as well as outputs from objective audio processing metrics and any algorithms disclosed herein.

The storage device 1030 can be capable of providing mass storage for the system 1000. In some implementations, the storage device 1030 can be a non-transitory computer-readable medium. The storage device 1030 can include, for example, a hard disk device, an optical disk device, a solid-date drive, a flash drive, magnetic tape, and/or some other large capacity storage device. The storage device 1030 can alternatively be a cloud storage device, e.g., a logical storage device including multiple physical storage devices distributed on a network and accessed using a network. In some implementations, the information stored on the memory 1020 can also or instead be stored on the storage device 1030.

The input/output device 1040 can provide input/output operations for the system 1000. In some implementations, the input/output device 1040 can include one or more of network interface devices (e.g., an Ethernet card or an Infiniband interconnect), a serial communication device (e.g., an RS-232 10 port), and/or a wireless interface device (e.g., a short-range wireless communication device, an 802.7 card, a 3G wireless modem, a 4G wireless modem, a 5G wireless modem). In some implementations, the input/output device 1040 can include driver devices configured to receive input data and send output data to other input/output devices, e.g., a keyboard, a printer, and/or display devices. In some implementations, mobile computing devices, mobile communication devices, and other devices can be used.

In some implementations, the system 1000 can be a microcontroller. A microcontroller is a device that contains multiple elements of a computer system in a single electronics package. For example, the single electronics package could contain the processor 1010, the memory 1020, the storage device 1030, and/or input/output devices 1040.

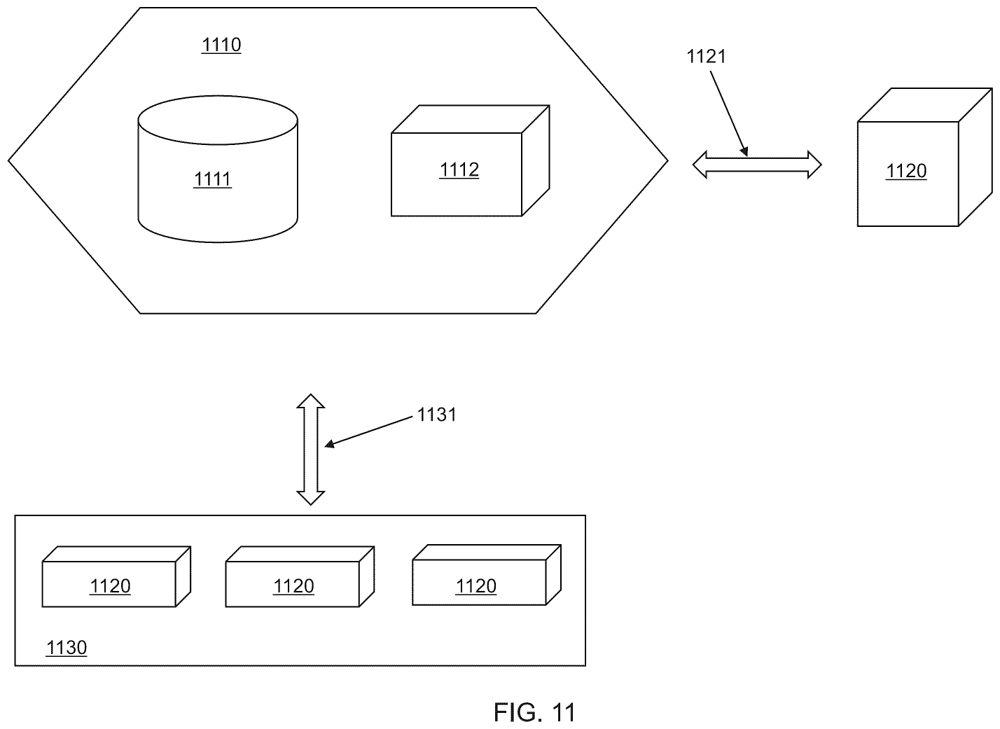

FIG. 11 is a block diagram of one exemplary embodiment of a cloud-based computer network 1110 for use in conjunction with the present disclosures. The cloud-based computer network 1110 can include a digital storage service 1111 and a processing service 1112, each of which can be provisioned by one or more individual computer processing and storage devices located in one or more physical locations. The cloud-based computer network 1110 can send and receive 1121, 1131, via the internet or other digital connection means, data from individual computer systems 1120 (e.g., a wearable armband device) as well as from networks 1130 of individual components 1120 (e.g., a plurality of wearable armband devices or a plurality of sensors or groups of sensors). The cloud-based computer network 1110 can facilitate or complete the execution of operations such as executing a prediction algorithm and conducting electronic communication with any of the devices disclosed herein, among other features described in conjunction with the present disclosure.

Although an example processing system has been described above, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine-readable storage device, a machine-readable storage substrate, a memory device, a composition of matter effecting a machine-readable propagated signal, or a combination of one or more of them.

Various embodiments of the present disclosure can be implemented at least in part in any conventional computer programming language. For example, some embodiments can be implemented in a procedural programming language (e.g., “C” or ForTran95), or in an object-oriented programming language (e.g., “C++”). Other embodiments can be implemented as a pre-configured, stand-along hardware element and/or as preprogrammed hardware elements (e.g., application specific integrated circuits, FPGAs, and digital signal processors), or other related components.

The term “computer system” can encompass all apparatus, devices, and machines for processing data, including, by way of non-limiting examples, a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Such implementation can include a series of computer instructions fixed either on a tangible, non-transitory medium, such as a computer readable medium. The series of computer instructions can embody all or part of the functionality previously described herein with respect to the system. Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

Those skilled in the art should appreciate that such computer instructions can be written in a number of programming languages for use with many computer architectures or operating systems. Furthermore, such instructions can be stored in any memory device, such as semiconductor, magnetic, optical, or other memory devices, and can be transmitted using any communications technology, such as optical, infrared, microwave, or other transmission technologies.

Among other ways, such a computer program product can be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the present disclosure may be implemented as a combination of both software (e.g., a computer program product) and hardware. Still other embodiments of the present disclosure are implemented as entirely hardware, or entirely software.

Various embodiments of the invention are provided throughout the present disclosure. For instance, the following numbered embodiments are contemplated and are non-limiting:
1. An armband device including a flexible strap and a single-sided electrocardiogram (SS-ECG) sensor.
2. The armband device of clause 1, any other suitable clause, or any combination of suitable clauses, where the armband device further includes at least one or more of the following: one or more bioimpedance (BioZ) sensors, one or more electrodermal activity (EDA) sensors, one or more photoplethysmography (PPG) sensors, one or more temperature sensors, an accelerometer, or a gyroscope.
3. The armband device of clause 2, any other suitable clause, or any combination of suitable clauses, where the housing compartment includes one or more of: the SS-ECG sensor, the BioZ sensor, the EDA sensor, the PPG sensor, the temperature sensor, the accelerometer, or the gyroscope,
4. The armband device of clause 1, any other suitable clause, or any combination of suitable clauses, where the armband device further includes one or more of the following:

data storage, a microcontroller, a Bluetooth module, a WiFi module, a battery, a charge circuit
5. A method of identifying or predicting a glycemic event in a subject, said method including the step of contacting the armband device of any of clauses 1 to 22 to the subject, where the armband device detects one or more signals that are computed to identify or predict the glycemic event.
6. The method of clause 5, any other suitable clause, or any combination of suitable clauses, where the contacting to the subject includes wearing the armband device on the upper arm of the subject.
7. The method of clause 5, any other suitable clause, or any combination of suitable clauses, where the glycemic event is a hypoglycemic event and/or a hyperglycemic event
8. The method of clause 5, any other suitable clause, or any combination of suitable clauses, where the one or more signals are computed using a prediction model to identify or predict the glycemic event.
9. The method of clause 5, any other suitable clause, or any combination of suitable clauses, where the method further includes delivering an alert to the subject of the glycemic event, data to the subject of the glycemic event, or both.

10. The method of clause 5, any other suitable clause, or any combination of suitable clauses, where the method further includes delivering an alert to a caregiver of the subject of the glycemic event, data to a caregiver of the subject of the glycemic event, or both.
11. The method of clause 5, any other suitable clause, or any combination of suitable clauses, where the method further includes delivering an alert to a healthcare provider of the subject of the glycemic event, data to a healthcare provider of the subject of the glycemic event, or both.
12. A wearable multi-sensor device for measuring physiological properties, the device including:
at least one sensor housing including a plurality of non-invasive sensors including:
   a single-sided electrocardiography sensor,
   a bioimpedance and electrodermal activity sensor, and
   a photoplethysmography sensor;
a fixation element configured to secure a skin-facing side of the at least one sensor housing to exposed skin of a user; and
a communication module configured to receive signals from the plurality of non-invasive sensors and output data from the device.
13. The device of clause 12, any other suitable clause, or any combination of suitable clauses, where the data is suitable for use in predicting glycemic events in the user.
14. The device of clause 12, any other suitable clause, or any combination of suitable clauses, where the plurality of non-invasive sensors further includes one or more of a skin temperature sensor and a motion sensor, the motion sensor configured to sense motion of the user wearing the device.
15. The device of clause 12, any other suitable clause, or any combination of suitable clauses, where the exposed skin of the user includes the user's upper arm.
16. The device of clause 15, any other suitable clause, or any combination of suitable clauses, where the fixation element and at least one sensor housing are configured to locate the bioimpedance and electrodermal activity sensor proximal to a brachial artery location of the user.
17. The device of clause 16, any other suitable clause, or any combination of suitable clauses, where the bioimpedance and electrodermal activity sensor includes at least one current-sensing electrode and at least one voltage-sensing electrode.
18. The device of clause 17, any other suitable clause, or any combination of suitable clauses, where the at least one current-sensing electrode and at least one voltage-sensing electrode are arranged to be placed in a line along a path of the brachial artery location.
19. The device of clause 12, any other suitable clause, or any combination of suitable clauses, where the photoplethysmography sensor includes a multi-wavelength LED array.
20. The device of clause 12, any other suitable clause, or any combination of suitable clauses, where the bioimpedance and electrodermal activity sensor includes a plurality of ECG electrodes.
21. The device of clause 20, any other suitable clause, or any combination of suitable clauses, where the at least one sensor housing includes a plurality of ECG electrode housings each carrying one of the plurality of ECG electrodes, the plurality of ECG electrode housings being spaced apart from each other.
22. A method of predicting glycemic events in a user, the method including: given at least one device worn by the user, the device including a plurality of non-invasive sensors positioned against skin of the user, the sensors including a single-sided electrocardiography sensor, a skin temperature sensor, a bioimpedance and electrodermal activity sensor, and a photoplethysmography sensor;
collecting physiological signals of the user from the plurality of non-invasive sensors;
generating data based on the physiological signals, the data being suitable for use in predicting glycemic events in the user; and
processing the data using a prediction model executed by a computer processor, the prediction model returning, as an output, an indication of an occurrence of a glycemic event or a likelihood of occurrence of a glycemic event of the user.
23. The method of clause 22, any other suitable clause, or any combination of suitable clauses, further including providing the indication to the user;
24. The method of clause 22, any other suitable clause, or any combination of suitable clauses, further including transmitting the data from the device to a second electronic device, the second electronic device conducting the processing of the data.
25. The method of clause 22, any other suitable clause, or any combination of suitable clauses, further including, before the processing of the data, conditioning the data and extracting a plurality of features from the data, the prediction model using the extracted features as an input to determine the indication.
26. The method of clause 25, any other suitable clause, or any combination of suitable clauses, further including, before the processing of the data, identifying one or more salient features from the plurality of features and identifying correlations between the one or more salient features, the prediction model using the identified one or more salient features and the identified correlations as an input to determine the indication.
27. The method of clause 26, any other suitable clause, or any combination of suitable clauses, further including, before the processing of the data, determining a suitable prediction model based on the one or more salient features.
28. The method of clause 25, any other suitable clause, or any combination of suitable clauses, where extracting a plurality of features from the data comprises extracting at least one of: heart rate variability measures, ECG, PPG, and BI beat morphology information, time-domain and frequency-domain from accelerometer and gyroscope data, skin temperature, or breathing waveforms.
29. The method of clause 25, any other suitable clause, or any combination of suitable clauses, where the plurality of features extracted is selected based on their potential salience in predicting a future hypoglycemic and/or hyperglycemic event based on a score that ranks their importance and potential.
30. The method of clause 22, any other suitable clause, or any combination of suitable clauses, where the indication is based on a predicted probability of a hypoglycemic and/or a hyperglycemic event calculated by the prediction model.
31. The method of clause 22, any other suitable clause, or any combination of suitable clauses, where the prediction model is configured to use calculate beat level hypoglycemia and Hyperglycemia predictions.
32. The method of clause 31, any other suitable clause, or any combination of suitable clauses, further including extracting beat morphology features from the physiological signals, and wherein the prediction model is configured to determine the indication as a function of the extracted morphology features.

One skilled in the art will appreciate further features and advantages of the disclosures based on the provided for descriptions and embodiments. Accordingly, the inventions are not to be limited by what has been particularly shown and described. All publications and references cited herein are expressly incorporated herein by reference in their entirety.

What is claimed is:

1. A wearable multi-sensor device for measuring physiological properties, the device comprising:
at least one sensor housing comprising a plurality of non-invasive sensors including:
a single-sided electrocardiography sensor,
a bioimpedance and electrodermal activity sensor, and
a photoplethysmography sensor;
a fixation element configured to secure a skin-facing side of the at least one sensor housing to exposed skin of a user; and
a communication module configured to receive signals from the plurality of non-invasive sensors and output data from the device.

2. The device of claim 1, wherein the data is suitable for use in predicting glycemic events in the user.

3. The device of claim 1, wherein the plurality of non-invasive sensors further includes one or more of a skin temperature sensor and a motion sensor, the motion sensor configured to sense motion of the user wearing the device.

4. The device of claim 1, wherein the exposed skin of the user includes the user's upper arm.

5. The device of claim 4, wherein the fixation element and at least one sensor housing are configured to locate the bioimpedance and electrodermal activity sensor proximal to a brachial artery location of the user.

6. The device of claim 5, wherein the bioimpedance and electrodermal activity sensor comprises at least one current-sensing electrode and at least one voltage-sensing electrode.

7. The device of claim 6, wherein the at least one current-sensing electrode and at least one voltage-sensing electrode are arranged to be placed in a line along a path of the brachial artery location.

8. The device of claim 1, wherein the photoplethysmography sensor comprises a multi-wavelength LED array.

9. The device of claim 1, wherein the bioimpedance and electrodermal activity sensor comprises a plurality of ECG electrodes.

10. The device of claim 7, wherein the at least one sensor housing comprises a plurality of ECG electrode housings each carrying one of the plurality of ECG electrodes, the plurality of ECG electrode housings being spaced apart from each other.

11. A method of predicting glycemic events in a user, the method comprising:
given at least one device worn by the user, the device comprising a plurality of non-invasive sensors positioned against skin of the user, the sensors including a single-sided electrocardiography sensor, a skin temperature sensor, a bioimpedance and electrodermal activity sensor, and a photoplethysmography sensor;
collecting physiological signals of the user from the plurality of non-invasive sensors;
generating data based on the physiological signals, the data being suitable for use in predicting glycemic events in the user; and
processing the data using a prediction model executed by a computer processor, the prediction model returning, as an output, an indication of an occurrence of a glycemic event or a likelihood of occurrence of a glycemic event of the user.

12. The method of claim 11, further comprising providing the indication to the user.

13. The method of claim 11, further comprising transmitting the data from the device to a second electronic device, the second electronic device conducting the processing of the data.

14. The method of claim 11, further comprising, before the processing of the data, conditioning the data and extracting a plurality of features from the data, the prediction model using the extracted features as an input to determine the indication.

15. The method of claim 14, further comprising, before the processing of the data, identifying one or more salient features from the plurality of features and identifying correlations between the one or more salient features, the prediction model using the identified one or more salient features and the identified correlations as an input to determine the indication.

16. The method of claim 15, further comprising, before the processing of the data, determining a suitable prediction model based on the one or more salient features.

17. The method of claim 14, wherein extracting a plurality of features from the data comprises extracting at least one of: heart rate variability measures, ECG, PPG, and BI beat morphology information, time-domain and frequency-domain from accelerometer and gyroscope data, skin temperature, or breathing waveforms.

18. The method of claim 14, wherein the plurality of features extracted is selected based on their potential salience in predicting a future hypoglycemic and/or hyperglycemic event based on a score that ranks their importance and potential.

19. The method of claim 11, wherein the indication is based on a predicted probability of a hypoglycemic and/or a hyperglycemic event calculated by the prediction model.

20. The method of claim 11, wherein the prediction model is configured to use calculate beat level hypoglycemia and Hyperglycemia predictions.

21. The method of claim 20, further comprising extracting beat morphology features from the physiological signals, and wherein the prediction model is configured to determine the indication as a function of the extracted morphology features.
